(19) United States
(12) Patent Application Publication
(10) Pub. No.: US 2006/0134213 A1
(43) Pub. Date: Jun. 22, 2006

(54) Stabilized ramipril compositions and methods of making

(76) Inventors: Edward S. Wilson, Cary, NC (US); Martin W. Beasley, Cary, NC (US)

Correspondence Address: JONES DAY, 222 EAST 41ST ST, NEW YORK, NY, 10017, US

(21) Appl. No.: 11/269,388

(22) Filed: Nov. 7, 2005

Related U.S. Application Data
(60) Provisional application No. 60/625,270, filed on Nov. 5, 2004.

(51) Int. Cl. A61K 9/24

(52) U.S. Cl. 424/472

(57) ABSTRACT

The present invention relates to ramipril compositions with improved stability. More particularly, the present invention is directed to pharmaceutical compositions comprising ramipril that are stabilized against decomposition into degradation products, namely, ramipril-diketopiperazine and ramipril-diacid, during formulation and storage conditions. The present invention also relates to methods for making and methods of manufacturing stabilized ramipril compositions.

This application claims the benefit of U.S. Provisional Application No. 60/625,270, filed Nov. 5, 2004 the contents of which are incorporated herein in its entirety.

### FIELD OF THE INVENTION

The present invention relates to novel pharmaceutical compositions comprising ramipril. More particularly, the compositions of the present invention have improved stability and are less susceptible to degradation relative to other compositions comprising ramipril. The present invention also relates to methods of making and methods of manufacturing such compositions.

### BACKGROUND

In general, drug stability is an important consideration during the design, manufacture and storage of pharmaceutical compositions. Drugs that lack stability can degrade into degradant products which can cause side effects or, in some cases, can cause a decrease in the efficacy and bioavailability of the drug itself, making it difficult for doctors to prescribe consistent and effective treatments.

One group of drugs that is susceptible to degradation is angiotensin-converting enzyme inhibitors, or ACE inhibitors. ACE inhibitors are encompassed in a class of drugs that was first introduced in about 1981. ACE inhibitors work by blocking the action of the ACE enzyme in human subjects and animals. The ACE inhibitors accomplish this blocking action by binding to the zinc component of the ACE enzyme.

Ramipril is an important ACE inhibitor used in the treatment of cardiovascular disease, especially hypertension, and it is one of the most frequently prescribed drugs for congestive heart failure. In hypertensive patients (herein patient and subject can be used interchangeably), ramipril is known to reduce peripheral arterial resistance causing a reduction in blood pressure without a compensatory rise in heart rate. Ramipril has also been shown to reduce mortality in patients with clinical signs of congestive heart failure after surviving an acute myocardial infarction. Ramipril may have an added advantage over many other ACE inhibitors due to its pronounced inhibition of the ACE enzymes in tissues resulting in organ protective effects in such organs as the heart, kidney, and blood vessels.

Even though ramipril is without question one of the most important ACE inhibitors available today, current ramipril formulations show a considerable degree of instability. The degradation of ramipril is believed to occur mainly via two pathways: (a) hydrolysis to ramipril-diacid; and (b) cyclization or condensation to ramipril-diketopiperazine, also referred herein as ramipril-DKP. These ramipril-diacid and ramipril-DKP compounds form, as indicated above, as a result of cyclization, condensation and/or breakdown arising from exposure to heat, air, moisture, stress, compaction or other interactions or events.

Currently, the leading formulation of ramipril is a capsule. This is primarily due to the fact that ramipril needs special care when formulating into pharmaceutical preparations due the physical stress associated with formulating processes which can increase the rate the decomposition of ramipril into degradant products. Indeed, factors that influence the stability of ramipril formulations are mechanical stress, compression, manufacturing processes, excipients, storage conditions, heat and moisture.

Attempts to overcome ramipril stability have been reported in PCT/EP2004/00456, PCT/CA2002/01379 and United States Published Application No. 2005/0069586.

PCT/EP2004/00456 describes a process to formulate ramipril compositions that utilizes excipients with low water content and processing parameters and packaging material that prohibit water or moisture uptake. Although the excipients include glyceryl behenate, microcrystalline cellulose and starch, PCT/EP2004/00456 does not teach pre-blending or co-milling the ramipril with glyceryl behenate or substantially coating the ramipril with glyceryl behenate. Moreover, the ramipril compositions taught have a high rate of ramipril-DKP formulation of 9.56% after two months at ambient temperature and humidity. Additionally, even when placed in air-tight packaging, the ramipril compositions have a rate of ramipril-DKP formation of 2.0%, after one month at 40° C. and at 75% humidity.

PCT/CA2002/01379 describes solid ramipril capsules that comprise a mixture of ramipril and lactose monohydrate as the diluent. According to PCT/EP2004/000456, the process includes lactose monohydrate as the major excipient to formulate ramipril compositions in an attempt to improve ramipril stability. However, immediately after formation of the described capsules, ramipril-DKP formation is already at 1.10%.

United States Published Application No. 2005/0069586 describes ramipril tablets that have an admixture of ramipril and sodium stearyl fumerate with reduced ramipril-DKP formation, but does not teach pre-blending or co-milling the ramipril with glyceryl behenate or substantially coating the ramipril with any blending agent.

Citations of any reference in the Background section of this application is not an admission that the reference is prior art to the application.

### SUMMARY OF THE INVENTION

The present invention is based in part on the discovery that stable oral dosage forms comprising ramipril can be achieved by first pre-blending or co-milling glyceryl behenate with ramipril during manufacture of ramipril tablets. The inventors have made the surprising discovery that by combining ramipril with glyceryl behenate, prior to formulation of ramipril into a dosage form, the rate of degradant production is extremely low. Without being limited to one particular theory, the inventors of the present invention believe that the glyceryl behenate coats the ramipril and is able to protect the ramipril from physical and environmental stress that, under normal conditions, cause the ramipril to degrade into degradant products such as ramipril-DKP and ramipril-diacid.

In particular, the inventors have demonstrated that by utilizing glyceryl behenate as a blending agent, ramipril decomposition into degradant products, such as ramipril-DKP and ramipril diacid, can be significantly reduced. Indeed, the inventors have demonstrated that the rate of decomposition of ramipril in compositions of the invention is less than 0.05% of the total weight of ramipril on average per month for at least 36 months from the date that the ramipril compositions are first formulated.

As such, the pharmaceutical compositions contemplated by the present invention comprise ramipril, wherein the ramipril has a low rate of degradation and is substantially free of ramipril-DKP and ramipril-diacid. Moreover, the pharmaceutical compositions of the present invention have increased stability, bioavailability and shelf-life compared to current formulations. Specifically, the inventors have shown that the compositions of the present invention have improved bioavailability compared to Altace® Additionally, the pharmaceutical compositions of the present invention allow ramipril to maintain potency, assuring health care providers and patients that they are giving and receiving consistent and exact treatment. The invention also contemplates reducing the rate of ramipril-DKP formation, especially under formulation and extended storage conditions.

The present invention also relates to methods of making the pharmaceutical compositions, of the present invention. Such methods comprise first pre-blending or co-milling ramipril with a blending agent. The methods of the present invention also comprise first coating ramipril with a blending agent prior to formulation of ramipril into a dosage form.

### BRIEF DESCRIPTION OF THE FIGURES

FIG. 1 shows a method of making the pharmaceutical compositions of the present invention.

FIG. 2 is a graph that illustrates a linear rate of ramipril-DKP formation of less than about 0.5% ramipril-DKP formation after a tested period of 3 months at room temperature and less about 2% ramipril-DKP formation after an extrapolated period of 36 months at room or ambient temperature from a ramipril tablet produced with coated ramipril particles.

FIG. 3 is a graph that illustrates a linear rate of ramipril- ramipril-DKP formation of less than about 0.5% ramipril-DKP formation after a tested period of 3 months at room temperature and less about 1.5% ramipril-DKP formation after an extrapolated period of 36 months at room or ambient temperature from a ramipril tablet produced with coated ramipril particles.

FIG. 4 is a graph that illustrates a linear rate of ramipril-DKP formation of less than about 0.5% ramipril-DKP formation after a tested period of 3 months at room temperature and less about 3% ramipril-DKP formation after an extrapolated period of 36 months at room or ambient temperature from a ramipril tablet produced with coated ramipril particles.

FIG. 5 is a graph that illustrates the rate of decomposition of ramipril in a formulation of the present invention compared to currently available formulations.

FIG. 6 is a graph that illustrates the rate of ramipril-DKP formation in 1.25 mg, 5 mg, 10 mg and 20 mg tablets made in accordance to the present invention.

### DETAILED DESCRIPTION

The terms “stabilized”, “stability”, “improved stability” or “stable” as applied to ramipril, can encompass products that are substantially free of breakdown products or degradants. Such products or degradants include, but are not limited to, ramipril-diacid and ramipril-DKP.

The term “substantially free” refers to the ramipril formulations described herein that have significantly reduced levels of detectable breakdown products; e.g., ramipril-diacid and/or ramipril-DKP.

The term “cardiovascular disorder(s)” as used herein broadly and encompasses any disease, illness, sickness, disorder, condition, symptom or issue involving or concerning any part or portion of the heart or blood vessels of an animal, including a human. The term “blood vessel”, as used herein, is defined to include any vessel in which blood circulates. Such cardiovascular disorders include, for example, arterial enlargements, arterial narrowings, peripheral artery disease, atherosclerotic cardiovascular disease, high blood pressure, angina, irregular heart rates, inappropriate rapid heart rate, inappropriate slow heart rate, angina pectoris, heart attack, myocardial infarction, transient ischemic attacks, heart enlargement, heart failure, congested heart failure, heart muscle weakness, inflammation of the heart muscle, overall heart pumping weakness, heart valve leaks, heart valve stenosis (failure-to-open fully), infection of the heart valve leaflets, heart stoppage, asymptomatic left ventricular dysfunction, cerebrovascular incidents, strokes, chronic renal insufficiency, and diabetic or hypertensive nephropathy. These above-listed conditions commonly arise in healthy, pre-disposed or critically ill patients, and may or may not be accompanied by hypertension, angina, light-headedness, dizziness, fatigue or other symptoms.

The terms “treat(s)”, “treated”, “treating” or “treatment” are used herein interchangeably and refer to any treatment of a disorder in an animal diagnosed or inflicted with such disorder and includes, but is not limited to: (a) caring for an animal diagnosed or inflicted with a disorder; (b) curing or healing an animal diagnosed or inflicted with a disorder; (c) causing regression of a disorder in an animal; (d) arresting further development or progression of a disorder in an animal; (e) slowing the course of a disorder in an animal; (f) relieving, improving, decreasing or stopping the conditions of a disorder in a animal; (g) relieving, decreasing or stopping the symptoms caused by or associated with a disorder in an animal; or (h) reducing the frequency, number or severity of episodes caused by or associated with a disorder in an animal.

The terms “prevent(s)”, “prevented”, “preventing” or “prevention” are used herein interchangeably and refer to any prevention or any contribution to the prevention of a disorder in an animal or the development of a disorder if none has occurred in an animal which may be predisposed to such disorder but has not yet been inflicted with or diagnosed as having such disorder.

The phrase “safe and effective amount(s)”, as used herein, means any amount of a drug which, when administered to a subject to be treated, will achieve a beneficial pharmacological effect or therapeutic improvement consistent with the objectives of the present invention without causing serious, adverse or otherwise treatment-limiting side effects (at a reasonable benefit/risk ratio), within the scope of sound medical judgment.

The term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range.

As used herein, the terms “comprising,” “comprises”, “comprised of,” “including,” “includes,” “included,” “involving,” “involves,” “involved,” and “such as” are used in their open, non-limiting sense.

It should be understood that the phrase “pharmaceutically acceptable” is used adjectivally herein to mean that the modified noun is appropriate for use in a pharmaceutical product.

The term “pharmaceutically acceptable salt” refers to a salt that retains the biological effectiveness of the free acid and/or base of the specified compound. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Mack Publ. Co., Easton.

The term “derivative” as used herein means a chemically modified compound wherein the chemical modification takes place at one or more functional groups of the compound and/or on an aromatic ring, when present. The derivative may or may not retain the pharmacological activity of the compound from which it is derived.

The term “pharmaceutical grade” as used herein, means that a substance meets pharmaceutical standards, and that its purity is superior as compared to the purity of the same such substance when classified as food grade, which is less pure.

The pharmaceutical compositions of the present invention relate to compositions comprising a drug that is susceptible to degradation when exposed to the environment or exposed to physical stresses during the manufacturing process and wherein the rate of degradation of the drug is extremely low.

On average, the rate of decomposition of the drug, in the pharmaceutical compositions of the present invention is between 0.00-0.09% of the total weight of the drug per month. Preferably the rate of decomposition of the drug in the compositions of the present invention is 0.04-0.05% of the total weight of the drug per month.

In various embodiments, the pharmaceutical compositions of the present invention result in rate of degradation of the drug between about 0.0-0.6% of the total weight of the drug during about the first three months after the compositions are formed and between about 0.0-0.4% of the total weight of the drug during a period of at least about 36 months after the pharmaceutical composition are formed.

In one embodiment, the rate of decomposition of the drug in the pharmaceutical compositions of the present invention is less than about 0.04% to about 0.095% of the total weight of the drug, at room temperature, on average per month for at least about 36 months from the date that the pharmaceutical compositions are first formulated. Preferred pharmaceutical compositions have a rate of decomposition of the drug of less than about 0.04% to about 0.085% of the total weight of the drug, at room temperature, on average per month for an extended period, more preferred pharmaceutical compositions have a rate of decomposition of the drug of less than about 0.04% to about 0. 055% of the total weights of the drug, at room temperature, on average per month for an extended period, and even more preferred pharmaceutical compositions have a rate of decomposition of the drug of less than about 0.04% to about 0.042% of the total weight of the drug, at room temperature, on average per month for an extended period.

Preferably the drug compositions of the present invention result in rate of degradation of the drug of less than about 0.3% during about the first three months of the total weight of the drug and less than about 2.0% of the total weight of the drug during a period of at least about 36 months after the first three month period. Preferred pharmaceutical compositions have a rate of degradation of the drug of less than about 0.3% of the total weight of the drug during about the first three months and less than about 1.5% of the total weight of the drug during a period of at least about 36 months after the first three month period.

The present invention encompasses pharmaceutical compositions that comprise a drug susceptible to degradation when exposed to the environment or exposed to physical stresses during the manufacturing process; and a blending agent.

In certain embodiments, the drug is an ACE inhibitor. Suitable ACE inhibitors include, but are not limited to, captopril, benazepril, enalapril, lisinopril, fosinopril, ramipril, perindopril, quinapril, moexipril, and trandolapril

Of the ACE inhibitors, ramipril, its derivatives and salts are of special interest. Suitable ramipril derivatives and salts include, but are not limited to, the esters and those common salts known to be substantially equivalent to ramipril. Suitable ramipril esters include, but are not limited to, hexahydroramipril, ramipril benzyl ester, isopropyl ester, ethyl ester or methyl ester. Pharmaceutically acceptable salts of ramipril include, but are not limited to, salts with pharmaceutically acceptable amines or inorganic or organic acids such as, HCl, HBr, H₂SO₄, maleic acid, fumaric acid, tartaric acid and citric acid.

Ramipril is a 2-aza-bicyclo [3.3.0]-octane-3-carboxylic acid derivative with five chiral centers, and 32 different enantiomeric forms. The chemical name of ramipril is (2S,3aS,6aS)-1[(S)-N-[(S)-1-carboxy-3-phenylpropyl]alanyl]octahydrocyclo-penta[b]pyrrole-2-carboxylic acid, 1-ethyl ester is most preferred and has the following chemical structure:

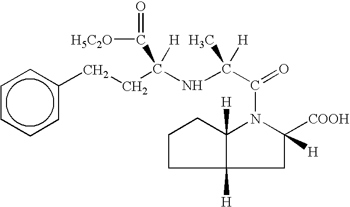

Ramipril is converted to ramiprilat in the body by hepatic cleavage of the ester group. Ramiprilat, the diacid or free acid metabolite of ramipril, is obtained in vivo upon administration of ramipril but ramiprilat is not absorbed in-vivo from the GI tract.

In preferred embodiments of the present invention the percent of ramiprilat does not exceed 20% after 8 weeks at 40° C. and 75% relative humidity. Preferably, the percent of ramiprilat does not exceed 1.0% during the life of the composition. Most preferably, the percent of ramiprilat does not exceed 0.5% during the life of the composition.

Ramipril is marketed in the United States under the brand name Altace® and abroad under the brand name Delix®. Altace® (ramipril) is supplied as hard shell capsules for oral administration containing 1.25 mg, 2.5 mg, 5 mg or 10 mg of ramipril.

Ramipril compositions of the present invention can be formulated with any form of ramipril known in the art. Ramipril suitable for the present invention can be uncoated or be coated with a coat forming material. Ramipril and processes for making and using ramipril are described and claimed in U.S. Pat. Nos. 4,587,258, 5,061,722 and 5,403,856, all of which are incorporated herein by reference in their entirety. The preparation of ramipril has also been described in EP 0 079 022 A2, EP 0 317 878 A1 and DE 44 20 102 A, which are incorporated herein by reference in their entirety.

Uncoated ramipril suitable for the present invention includes ramipril, as obtained from the Aventis Pharma Deutschland GmbH (Frankfurt on Main, Germany). Coated ramipril suitable for the present invention can be any coated ramipril known in the art. For example, coated ramipril suitable for the present invention can include ramipril particles that are coated with a suitable coat forming material. Coated ramipril suitable for the present invention can be partially, substantially or completely covered with a coat forming material. Ramipril particles can include but are not limited to, coated ramipril micro- or nanoparticles, coated ramipril crystalline particles, coated individual ramipril crystals and coated ramipril agglomerates, granules or beads. One preferred type of ramipril agglomerates is the GEcoated ramipril agglomerates, manufactured by Aventis Pharma Deutschland GmbH (Frankfurt on Main, Germany). Such GEcoated ramipril agglomerates are ramipril agglomerates coated with a hydroxypropyl methylcellulose polymer coating (1.192 mg GEcoated granules =1.0 mg ramipril). Coated ramipril particles, suitable for the present invention, can also be made according to the methods disclosed in U.S. Pat. Nos. 5,061,722; 5,151,433; 5,403,856; and 5,442,008, U.S. Provisional Application No. 60/625,270 and a Co-pending U.S. Application No. filed Nov. 7, 2005 (serial no. not yet assigned), herein incorporated by reference. The compositions of the present invention can also contain anhydrous, pharmaceutical grade ramipril powder comprising coated ramipril particles.

In preferred embodiments, the pharmaceutical compositions of the present invention, comprises ramipril wherein the ramipril is substantially stable against decomposition into degradant products, such as ramipril-diacid and ramipril-DKP. Additionally, the ramipril compositions of the present invention have improved stability and shelf-life. This improved stability allows the ramipril compositions to maintain potency and improve effectiveness and bioavailability of ramipril compared to other ramipril formulations.

The rate of decomposition of ramipril to ramipril-DKP, in the compositions of the present invention is between 0.00-0.09% of the total weight of ramipril per month. Preferably the rate of decomposition of ramipril in the compositions of the present invention is 0.04-0.05% of the total weight of ramipril per month.

For example, the ramipril compositions of the present invention result in ramipril-DKP formation of between 0.0-0.6% of the total weight of ramipril during about the first three months after the compositions are formed and between 0-4% of the total weight of ramipril during a period of at least about 36 months after the composition are formed.

In one embodiment, the pharmaceutical compositions of the present invention have a rate of decomposition of ramipril of less than about 0.04% to about 0.095% of the total weight of ramipril at room temperature, on average per month for at least about 36 months from the date that the ramipril compositions are first formulated. Preferred pharmaceutical compositions have ramipril-DKP formation of less than about 0.04% to about 0.085% of the total weight of ramipril at room temperature, on average per month for an extended period, more preferred the pharmaceutical compositions have ramipril-DKP formation of less than about 0.04% to about 0.055% of the total weight of ramipril at room temperature, per month on average for such an extended period, and even more preferred the pharmaceutical compositions have ramipril-DKP formation of less than about 0.04% to about 0.042% of the total weight of ramipril at room temperature, per month on average for an extended period of time.

Preferably the ramipril compositions of the present invention result in ramipril-DKP formation of less than about 0.3% during about the first three months of the total weight of ramipril and less than about 2.0% of the total weight of ramipril during a period of at least about 36 months after the first three month period. Preferred ramipril compositions result in ramipril-DKP formation of less than about 0.3% of the total weight of ramipril during about the first three months and less than about 1.5% of the total weight of ramipril during a period of at least about 36 months after the first three month period.

In one preferred embodiment, the compositions of the present invention comprise ramipril, wherein the rate ramipril decomposition to ramipril-DKP, is less than about 0.3% of the total weight of the ramipril during about the first three months after the compositions are formed.

In another preferred embodiment, the compositions of the present invention comprise ramipril, wherein the rate of ramipril decomposition to ramipril-DKP, is less than about 0.75% of the total weight of the ramipril during about the first 6 months after the compositions are formed.

In yet another preferred embodiment, the compositions of the present invention comprise ramipril, wherein the rate ramipril decomposition to ramipril-DKP, is less than about 3.0% of the total weight of the ramipril during about the first 36 months after the compositions are formed.

The blending agent can be any substance suitable for pre-blending and co-milling, which stabilizes the drug and significantly reduces the degradation of the drug. The phrase “blending agent” is interchangeable with “blending compound”. Preferably, the blending agent can coat the ramipril and reduce the degradation rate.

Blending agents contemplated by the present invention include polymers, starches, stearates, silicas, waxes (atomized glyceryl palmitostearate, dioctyl sodium sulphosuccinate), surfactants, and fatty acids (preferably having a chain length of eight carbons or greater which may contain one or more double bonds). For example, blending agents suitable for the present invention include, but are not limited to, include long chain fatty acid-containing glycerol esters. Blending agents include, but are not limited to, glyceryl behenate, glyceryl stearate, stearyl alcohol, magnesium stearate, macrogol stearate ether, palmitostearate, ethylene glycol, polyethylene glycol, ethylene oxide polymers, sodium lauryl sulfate, magnesium lauryl sulfate, sodium oleate, sodium stearyl fumerate, leucine, stearic acid, cetyl alcohol, lauryl alcohol, amylopectin, poloxymer or combinations thereof. Most preferably, the blending agent is glyceryl behenate.

The blending agent can be present from at least about 0.1 wt % and above by weight of the total composition. In a specific embodiment, the blending agent is present at about 0.5 wt. % and above. In another specific embodiment, the blending agent is present at about 1.0 wt. % and above. In another specific embodiment, the blending agent is present at about 2.0 wt. % and above. In a specific and preferred embodiment, the blending agent is present at about 3.0 wt. % and above. In another specific embodiment, the blending agent is present at about 4.0 wt. % and above (e.g., 5 and 10 wt. %).

The blending agent can be present from at least 0.1 wt % and above by weight of the total composition. In a specific embodiment, the blending agent is present at 0.5 wt. % and above. In another specific embodiment, the blending agent is present at 1.0 wt. % and above. In another specific embodiment, the blending agent is present at 2.0 wt. % and above. In a specific and preferred embodiment, the blending agent is present at 3.0 wt. % and above. In another specific embodiment, the blending agent is present at 4.0 wt. % and above (e.g., 5 and 10 wt. %).

Additionally, the blending agent can be present in a ratio of about 1:10 to about 10:1 of the drug. The blending agent can be present in a ratio of about 1:5 to about 5:1 or about 1:2 or 2:1 of the drug.

In yet another embodiment, the pharmaceutical compositions of the present invention comprise a drug and a blending agent, wherein the drug is coated by the blending agent. The drug can be substantially coated by the blending agent. The drug is substantially coated when the blending agent coats the drug wherein the drug has a low or no rate of degradation. For example, the drug can be between about 50% to 100% coated by the blending agent. Preferably, the drug is between about 75% to 100% coated by the blending agent or more preferably between about 85% to 100% coated by the blending agent. Most preferably, the drug is between about 95% to 100% coated by the blending agent.

In yet another embodiment, the pharmaceutical compositions of the present invention comprise a drug and a blending agent, wherein the drug is coated by the blending agent. The drug can be substantially coated by the blending agent. The drug is substantially coated when the blending agent coats the drug wherein the drug has a low or no rate of degradation. For example, the drug can be between 50% to 100% coated by the blending agent. Preferably, the drug is between 75% to 100% coated by the blending agent or more preferably between 85% to 100% coated by the blending agent. Most preferably, the drug is between 95% to 100% coated by the blending agent.

Pharmaceutical compositions of the present invention may also include pharmaceutically acceptable additives into any suitable type of unit dosage form. Suitable additives include, but are not limited to, diluents, binders, vehicles, carriers, excipients, binders, disintegrating agents, lubricants, swelling agents, solubilizing agents, wicking agents, cooling agents, preservatives, stabilizers, sweeteners, flavors, polymers etc. While any pharmaceutically acceptable additive is contemplated by the present invention, it should be understood that the additive(s) selected for compounding with coated ramipril particles should not defeat the stability objectives of the present invention. Even though some pharmaceutically acceptable additives may cause ramipril to degrade, such additives may be suitable for the present invention so long as such additives do not cause ramipril, as it is combined with a blending agent, to degrade.

Examples of excipients include, but are not limited to, acacia, alginic acid, croscarmellose, gelatin, gelatin hydrosylate, mannitol, plasdone, sodium starch glycolate, sorbitol, sucrose, and xylitol. For molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of additional stabilizers or preservatives include, but are not limited to, parahydroxybenzoic acid alkyl esters, antioxidants, antifungal agents, and other stabilizers/preservatives known in the art.

Examples of coloring agents include, but are not limited to, water soluble dye, Aluminum Lake, ion oxide, natural colors, titanium oxide, and the like.

Examples of diluents or fillers include, but are not limited to, water-soluble and/or water-insoluble tabletting fillers. The water-soluble diluent agent may be constituted from a polyol of less than 13 carbon atoms, in the form of directly compressible material (the mean particle size being between about 100 and about 500 microns), in the form of a powder (the mean particle size being less than about 100 microns) or a mixture thereof. The polyol is preferably chosen from the group comprising of mannitol, xylitol, sorbitol and maltitol. The water-insoluble diluent agent may be a cellulosic derivative, such as, microcrystalline cellulose or a starch, such as, pre-gelatinized starch. Especially preferred diluents are those with minimal moisture content, such as lactose monohydrate and magnesium oxide.

Examples of disintegrating agents include, but are not limited to, cross-linked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearyl fumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A portion of the lubricant may be used as an internal solid lubricant which is blended and granulated with other components of the granulation. Another portion of the lubricant may be added into the final blended material just before compression or encapsulation that coats the outside of the granules in the final blend.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Examples of polymers include, but are not limited to, polysaccharides, celluloses, and organic moieties such as polyvinyl pyrrolidines and plastics.

Examples of celluloses include, but are not limited to, hydroxypropylcellulose, hydroxypropylmethylcellulose, hydroxylpropyl-methylcellulose, hydroxyethylcellulose, ethylcellulose, cellulose acetate phthalate, cellulose acetate, polyvinyl acetate phthalate, polyvinylpyrrolidone, gelatin, hydroxypropyl methyl cellulose acetate succinate, hydroxypropyl methyl cellulose succinate, hydroxylpropyl cellulose acetate succinate, hydroxyethyl methyl cellulose succinate, hydroxyethyl cellulose acetate succinate, hydroxypropyl methyl cellulose phthalate, hydroxethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methylcellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimelllitate succinate, cellulose propionate trimellitate, cellulose butryrate trimellitate, cellulose acetate terephthalate, cellulose acetate isophthalate, cellulose acetate pyridine dicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid, cellulose acetate, ethyl picolinic acid cellulose acetate.

Other polymers that may be suitable for use with the present invention include, but are not limited to, acrylate and methacrylate copolymers. Exemplary commercial grades of such copolymers include the EUDRAGIT® series, which are copolymers of methacrylates, acrylates, carboxylic acid-functionalized vinyl polymers, such as the carboxylic acid functionalized polymethacrylates and carboxylic acid functionalized polyacrylates, amine-functionalized polyacrylates and polymethacrylates; proteins such as gelatin and albumin, and carboxylic acid functionalized starches such as starch glycolate, carboxylic acid functionalized polymethyacrylates, carboxylic acid functionalized polyacrylate, amine-functionalized polyacrylates, amine-functionalized polymethacrylates, proteins, carboxylic acid functionalized starches, vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, polyoxyethylene-polyoxypropylene copolymers, alkylacyloxy-containing repeat units, or cyclicamido-containing repeat units; polyvinyl alcohols that have at least a portion of their repeat units in the unhydrolyzed form; polyvinyl alcohol polyvinyl acetate copolymers; polyethylene glycol, polyethylene glycol polypropylene glycol copolymers, polyvinyl pyrrolidone polyethylene polyvinyl alcohol copolymers, and polyoxyethylene-polyoxypropylene block copolymers.

The flavouring may be advantageously chosen to give a combination of fast onset and long-lasting sweet taste and get a “round feeling” in the mouth with different texturers or additives. Cooling agents can also be added in order to improve the mouth feeling and provide a synergy with flavours and sweetness. Various other materials may be present as coatings or to otherwise modify the physical form of the dosage unit. For instance, tablets or capsules may be coated with shellac, sugar or both.

Additional illustrations of adjuvants which may be incorporated in the tablets include, but are not limited to, a binder such as gum tragacanth (arabic), acacia, corn starch, potato starch, alginic acid, povidone, acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, lactose monohydrate, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminium silicate, maltodextrin, methylcellulose, hydroxypropylcellulose, polyethylene, polyethylene oxide, polymethacrylates, plasdone, sodium alginate, starch, pregelatinized starch, zein or the like; a sweetening agent such as sucrose, potassium acesulfame, aspartame, lactose, dihydrochalcone neohesperidine, saccharin, sucralose, polyols such as xylitol, mannitol, and maltitol, sodium saccharide, Asulfame-K, Neotame®, glycyrrhizin, malt syrup and combinations thereof; a flavoring such as berry, orange, peppermint, oil of wintergreen, cherry, citric acid, tartaric acid, menthol, lemon oil, citrus flavor, common salt, and other flavors known in the art.

Pharmaceutical compositions of the present invention can be administered orally or internally to subjects. This can be accomplished, for example, by administering to the subject a solid or liquid oral dosage form by mouth or via a gastric feeding tube, a duodenal feeding tube, a nasogastric (ng) tube, a gastrostomy, or other indwelling tubes placed in the GI tract. Other forms of the drug may be in suppositories, suspensions, liquids, powders, creams, transdermal patches, and depots.

Oral pharmaceutical compositions of the present invention are generally in the form of individualized or multi unit doses, such as tablets, caplets, powders, suspension tablets, chewable tablets, rapid melt tablets, capsules, e.g., a single or double shell gelatin capsule, tablet-filled capsules, effervescent powders, effervescent tablets, pellets, granules, liquids, solutions, or suspensions, respectively.

While the present invention contemplates any solid dosage form suitable for oral administration, ramipril tablets, capsules, tablet-filled capsules and caplets are especially preferred. When the pharmaceutical compositions of the present invention are formed into tablets or caplets, it is to be understood that the tablets or caplets may be scored, and that they may be of any suitable shape and size, such as round, square, rectangular, oval, diamond, pentagon, hexagon or triangular, so long as the objectives of the present invention are not defeated. It is to be further understood that when tablet-filled capsules are selected, the tablets utilized therewith may be formed into shapes that either (a) correspond to the capsules to permit over-coating or encapsulation via the capsules or (b) readily fit inside the capsules.

The oral pharmaceutical compositions may contain a drug in any therapeutically effective amount, such as from about 0.001 mg or less to about 200 mg or more, or preferably from about 0.01 mg to about 100 mg or preferably from about 0.1 mg to about 50 mg. Preferably, the dosage range will be between about 1.25 mg to about 25 mg per patient per day; more preferably about 10 mg to about 20 mg per patient per day, and most preferably about 10 mg or 20 mg per day.

By way of example, a particularly preferred stabilized oral unit dose or composition of the present invention may contain ramipril in a dosage amount of about 1.25 mg, about 2.5 mg, about 5 mg, about 7.5 mg, about 10 mg, 12.5 mg, about 15 mg, about 20 mg, about 25 mg, about 30 mg, about 40 mg, about 50 mg, about 60 mg, about 70 mg, about 75 mg, about 80 mg, about 90 mg, or about 100 mg. Of course, it should be appreciated that a particular unit dosage form and amount can be selected to accommodate the desired frequency of administration used to achieve a specified daily dosage and therapeutic effect.

Of particular interest are stabilized 1.25, 2.5, 5, 10, 15 and 20 mg ramipril tablets, stabilized 1.25, 2.5, 5, 10, 15 and 20 mg ramipril caplets, stabilized 1.25, 2.5, 5, 10, 15 and 20 mg ramipril capsules and stabilized 1.25, 2.5, 5, 10, 15 and 20 mg ramipril tablet-filled capsules.

Consistent with the present invention, these and other dosage forms discussed herein may be administered to individuals on a regimen of one, two or more doses per day, at any time of the day.

The dosage of active ingredient in the compositions of the invention may be varied; however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets being followed by a patient, concurrent medication, and other factors, recognized by those skilled in the art. Based upon the foregoing, precise dosages depend on the condition of the patient and are determined by discretion of a skilled clinician. Generally, ramipril daily dosage levels of between about 0.010 to about 1.5 mg/kg of body weight are administered daily to mammalian patients, e.g., humans having a body weight of about 70 kg. The ramipril dosage range will generally be about 1.25 mg to 50 mg per patient per day, administered in single or multiple doses.

Nonetheless, it should be understood that safe and effective amounts of ramipril utilized in accordance with the present invention will vary with the particular cardiovascular disorder, conditions and/or symptoms being treated, the age, weight and physical conditions of the subjects being treated, the severity of the cardiovascular disorder, conditions and/or symptoms, the duration of treatments, the nature of concurrent therapies, the specific dosage form employed, the particular pharmaceutically acceptable carriers utilized, and like factors within the knowledge and expertise of the attending physicians. Exemplary safe and effective amounts of ramipril include those amounts mentioned herein, administered one or more times per day, as will be more fully describe herein below.

The present invention is also generally directed towards methods of making pharmaceutical compositions with improved stability, bioavailability and shelf-life. The following methods of making a pharmaceutical compositions in accordance with the present can be used with any drug. Specifically, the methods of the present invention are directed to making pharmaceutical compositions comprising any drug that is susceptible to degradation when exposed to the environment or exposed to physical stresses during the manufacturing process.

The pharmaceutical compositions of the present invention can be made by first combining a drug with a blending agent so that the drug is coated with a blending agent before being processed into tablets. Combining the drug with the blending agent can be accomplished by blending, mixing, milling or co-milling, compressing, granulating, suspending, dissolving or precipitating the drug and the blending agent together.

Preferably, the combined drug and blending agent is suitable for use in preparing dosage forms by processes including, but not limited to, dry blend, direct compression formulations and hot melt extrusion processes.

Preferably, the methods of the present invention comprise an ACE inhibitor and more preferably, ramipril.

Methods of the present invention can comprise combining ramipril with a blending agent. Such methods can also further comprise adding an additive such as, but not limited to, a polymer, diluent, disintigrant or a combination thereof, before of after the ramipril is combined with the blending agent. Combining ramipril with the blending agent can be accomplished by blending, mixing, milling or co-milling, compressing, granulating, suspending, dissolving or precipitating the drug and the blending agent together.

In various embodiments, the invention contemplates methods comprising combining a blending agent and ramipril before the ramipril is further processed into tablets. Preferably, the blending agent and ramipril are pre-blended or co-milled before the ramipril is further processed into tablets. The invention also contemplates methods that further comprise adding additives including, but not limited to, diluents, binders, vehicles, carriers, excipients, binders, disintegrating agents, lubricants, swelling agents, solubilizing agents, wicking agents, cooling agents, preservatives, stabilizers, sweeteners, flavors, polymers, to the pre-blended or co-milled ramipril and blending agent.

In preferred embodiments methods of the present invention comprise first co-milling ramipril with a blending agent. Such methods can also further include additional steps comprising combining the co-milled ramipril and blending agent along with a polymer, diluent, disintigrant or a combination thereof.

In other preferred embodiments the methods of the present invention comprise pre-blending ramipril with a blending agent and then co-milling the ramipril and the blending agent. Such methods can also further include additional steps comprising combining the co-milled ramipril and blending agent along with a polymer, diluent, disintigrant or a combination thereof.

In other embodiments the methods of the present invention comprises blending ramipril with a blending agent; co-milling the ramipril and the blending agent and then re-blending the ramipril with the blending agent. Such methods can also further include additional steps comprising combining the ramipril and blending agent along with a polymer, diluent, disintigrant or a combination thereof.

In yet other embodiments, the method of the present invention comprises blending ramipril with a polymer and co- milling the ramipril and polymer with a blending agent. Such methods can also further include additional steps comprising combining the ramipril with a second polymer, diluent, disintigrant or a combination thereof, before or after being co-milled with the blending agent.

In one embodiment the method of making solid oral ramipril pharmaceutical compositions comprises blending coated ramipril with a blending agent; co-milling the coated ramipril and the blending agent; and re-blending the coated ramipril with a blending agent. Additionally, a polymer, a diluent, a lubricant or a disintigrant can be combined with the ramipril before or after being milled.

In the above methods, one purpose of the pre-blending and co-milling the blending agent and ramipril before the ramipril is further processed into tablets is to facilitate coating the ramipril with the blending agent. In all of the above methods the blending agent coats the ramipril. Preferably the blending agent coats between 50% to 100% of the ramipril, or between 75% to 100%, or between 85% to 100% and most preferably between 95% to 100%. Also in all of the above methods the preferred blending agent is glyceryl behenate

In a particularly preferred embodiment, ramipril and glycerol behenate are first co-milled, then followed by additional steps wherein, sodium stearyl fumarate and croscarmellose sodium are added to the ramipril and glycerol behenate blend.

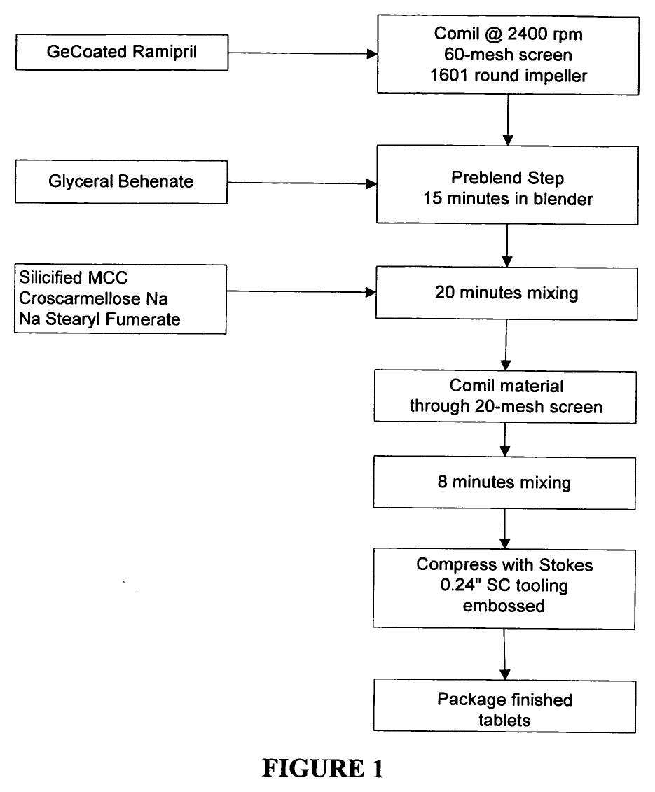

FIG. 1 shows one method of making pharmaceuticals of the present invention comprising GECoated ramipril. GEcoated ramipril is pre-milled though a 60-mesh screen. The milled ramipril is then pre-blended with glyceryl behenate for 15 minutes in a blender that has been grounded to reduce electrostatic charges. Croscarmellose sodium, sodium stearyl fumerate and silicified microcrystalline cellulose are added to the mixture and mixed for another 20 minutes. The co-milled mixture is then passed through a 20-mesh sieve. The sieved mixture is then placed into blender and mixed for an additional 8 minutes. The mixture is then compressed with a tablet press. The tablets finished tablets then can be packaged.

This process can be scaled, for example, to about 6 kg, in a 16-quart V-shell PK blender, and larger as needed. Tablets can be produced with a Fette P1200 24-station press, or similar equipment.

In the alternative, pharmaceutical compositions made by the above process can be formulated with uncoated ramipril as well. Also, microcrystalline cellulose can be replaced with diluents and fillers including but not limited to Ceolus®, lactose, anhydrous lactose, lactose monohydrate, starch, spray-dried mannitol (Pearlitol 200 SD), Prosolv® SMCC 90, or a combination thereof. Also, glyceryl behenate can be replaced with magnesium stearate.

The method, as shown in FIG. 1, can be used with any type of ramipril. Also, the mixing times and other parameters of the process can be varied to achieve the pharmaceutical compositions of the present invention comprising ramipril, wherein the ramipril has a low rate of degradation compared to current formulations.

An article of manufacture, as contemplated by the present invention, comprises a container holding a pharmaceutical composition suitable for oral administration of stabilized ramipril in combination with printed labeling instructions providing a discussion of when a particular dosage form should be administered.

The composition will be contained in any suitable container capable of holding and dispensing the dosage form and which will not significantly interact with the composition and will further be in physical relation with the appropriate labeling advising that a dosage form is more stable and bioavaliable with extended shelf life.

The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

The compositions, of the present invention, comprising coated ramipril can be administered to a subject for the treatment of cardiovascular disorders. Cardiovascular disorders include but are not limited to, hypertension, heart failure, congestive heart failure, myocardial infarction, atherosclerotic cardiovascular disease, asymptomatic left ventricular dysfunction, chronic renal insufficiency, and diabetic or hypertensive nephropathy.

The examples throughout herein and that follow are provided solely to illustrate representative embodiments of the invention. Accordingly, it should be understood, that the invention is not to be limited to the specific conditions or details described in these or any other example discussed herein, and that such examples are not to be construed as limiting the scope of the invention in any way. Throughout the specification, any and all references are specifically incorporated herein by reference in their entireties.

### EXAMPLES

### Example 1

Ramipril tablets were made with individually spray coated ramipril and GECoated ramipril according to the formulations shown in Tables 1 and 2.

TABLE 1

| | Formula | | | |
|---|---|---|---|---|
| | Compositions | III | I | II |
| (a) | Individually | about 1.49% | about 2.98% | about 2.98% |
| | Spray Coated | | | |
| | Ramipril particles | | | |
| | (HPMC) | | | |
| (b) | Microcrystalline | about 92.41% | about 93.02% | about 94.92% |
| | cellulose | | | |
| | (Prosolv ® | | | |
| | SMCC 50) | | | |
| (c) | glyceryl behenate | about 4.0% | about 2.0% | — |
| (d) | sodium stearyl | about 0.1% | — | about 0.1% |
| | fumarate | | | |
| | (PRUV ™) | | | |
| (d) | croscarmellose | about 2.0% | about 2.0% | about 2.0% |
| | sodium | | | |

TABLE 2

| | Formula | | | |
|---|---|---|---|---|
| | Compositions | I | II | III |
| (a) | GECoated | about 1.49% | about 2.98% | 11.92% |
| | Ramipril | | | |
| (b) | Microcrystalline | about 92.41% | about 90.92% | about 81.98% |
| | cellulose | | | |
| | (Prosolv ® | | | |
| | SMCC 50) | | | |
| (c) | glyceryl behenate | about 4.0% | about 4.0% | about 4.0% |
| (d) | sodium stearyl | about 0.1% | about 0.1% | about 0.1% |
| | fumarate | | | |
| | (PRUV ™) | | | |
| (d) | croscarmellose | about 2.0% | about 2.0% | about 2.0% |
| | sodium | | | |

The above tablets were made by pre-blending microcrystalline cellulose with the ramipril and then adding glyceryl behenate, sodium stearyl fumarate and croscarmellose sodium in a 16-quart V-shell blender and blending for about 20 min, then mill-blending the mixture through a Quadro Co-mil. The mixture was transferred to a 16-quart container and mixed for about 8 min, then compressed on a Stokes B2 tablet press, tooled with 16 stations with ¼″ standard concave (about 100 mg tablet weight) or 5/16″ standard concave (about 200 mg tablet weight) double-sided debossed tooling at about 48 rpm.

### Example 2

The following tablets were made according to the formulation in Table 3 by pre-milling GECoated ramipril through a 40 or 60 mesh screens and then pre-blended with a blending agent such as, glyceryl behenate, sodium stearyl fumerate or both. Silicified microcrystalline cellulose and croscarmellose sodium were added add mixed for an additional period of time. The mixture was co-milled through a 20 mesh screen and blended. The mixture was compressed into tablets.

Stability data measure by label claim (LC %) and DKP formation (DKP %) are shown in Tables 4 and 5. Sample testing was done at room temperature conditions (25 degrees C. and 60% humidity) and accelerated degradation conditions (40 degrees C. and 75% humidity).

TABLE 3

| | | | | sodium | sodium | |
|---|---|---|---|---|---|---|
| Sample # | | Glyceryl | Microcrystalline | stearyl | carboxymethyl- | |
| 100 mg | GECoated | Behenate | cellulose | fumarate | cellulose | Co- |
| Tablets | Ramipril | (Compritol) | (Prosolve) | (PRUV ™) | (Ac-di-Sol) | milled |
| 58F60A | 1.49% | 4% | 92.41% | 0.1% | 2.0% | 40 mesh |
| 59F61A | 1.49% | 2% | 94.41% | 0.1% | 2.0% | 40 mesh |
| 60F62A | 1.49% | 2% | 94.51% | 0% | 2.0% | 40 mesh |
| 61F63A | 1.49% | 0% | 94.61% | 0.1% | 2.0% | 40 mesh |
| 73F74A | 1.49% | 4% | 92.41% | 0.1% | 2.0% | 60 mesh |
| 74F75A | 1.49% | 2% | 94.41% | 0.1% | 2.0% | 60 mesh |
| 75F76A | 1.49% | 4% | 92.41% | | 2.0% | 40 mesh |

For samples 58F60A and 73F74A, GECoated ramipril was preblended with 4% glyceryl behenate and sodium stearyl fumarate.

For samples 59F61A and 74F75A, GECoated ramipril was preblended with 2% glyceryl behenate and sodium stearyl fumarate.

For samples 60F62A and 75F76A, GECoated ramipril was preblended with 2% glyceryl behenate.

For sample 61F63A, GECoated ramipril was preblended with sodium stearyl fumerate.

TABLE 4
Coated Ramipril Co-milled ˜40 mesh

| Sample | | | | | | | |
|---|---|---|---|---|---|---|---|
| # | Strength | | 2 wk 40/75 | 4 wk 40/75 | 8 wk 40/75 | 12 wk 40/75 | 12 wk RT |
| | | % LC | | | | | |
| | | Initial | | | | | |
| 58F60A | 1.25 mg | 107.4 | 108.7 | 108 | 104.6 | 104.5 | 108.5 |
| 59F61A | 1.25 mg | 104.6 | 108.2 | 107.3 | 106.6 | 104.1 | 107.9 |
| 60F62A | 1.25 mg | 104.5 | 103.1 | 104.5 | 102 | NT | NT |
| 61F63A | 1.25 mg | 106.2 | 103.8 | 105.6 | 99.1 | NT | NT |
| | | % DKP | | | | | |
| | | Initial | | | | | |
| 58F60A | 1.25 mg | 0.29 | 0.54 | 0.91 | 1.66 | 2.30 | 0.4 |
| 59F61A | 1.25 mg | 0.28 | 0.57 | 0.99 | 1.88 | 2.66 | 0.42 |
| 60F62A | 1.25 mg | 0.27 | 0.55 | 0.92 | 1.84 | NT | NT |
| 61F63A | 1.25 mg | 0.27 | 0.54 | 0.96 | 1.86 | NT | NT |

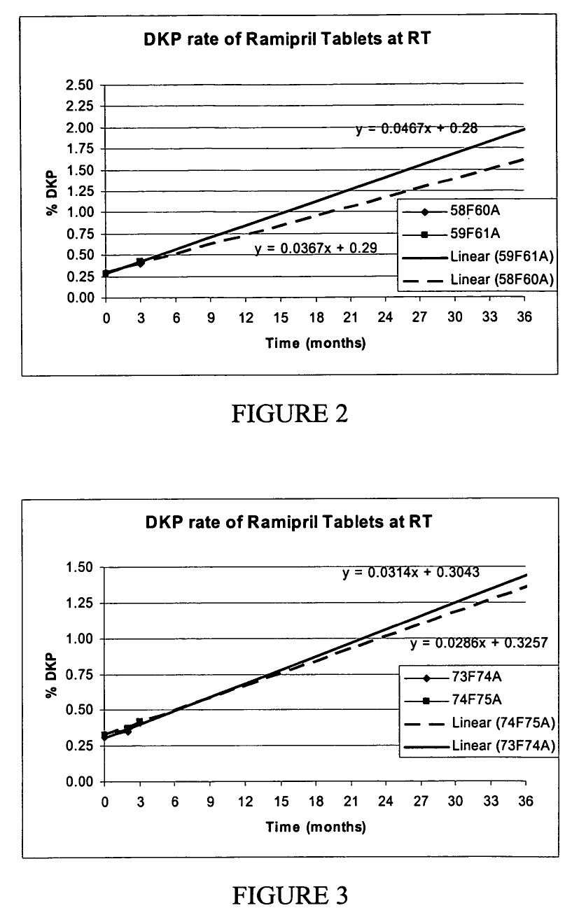

Long-term stability of ramipril tablets at room temperature is described. Ramipril-DKP rate up to about 36 months is shown in FIGS. 2-4. Ramipril-DKP formation is less than about 0.05% after 3 months and less than an extrapolated amount of about 3.0% after about 36 months in the examples tested. In addition to ramipril-DKP formation other degradation pathways for ramipril exist, including formation of ramiprilat (ramipril diacid). Premature formation (before patient administration) of ramiprilat is undesirable because it is not absorbed by the patient, and is therefore insufficiently bioavailability. Preferably, stability analyses should include detection of levels of ramiprilat.

FIG. 2 represents a linear regression of the rate of DKP formulation of samples 58F60A and 59F61A. 58F60A is represented by the dashed line and the rate of DKP % is represented by the equation y=0.0367x+0.29. 59F61A is represented by the solid line and the rate of DKP % is represented by the equation y=0.0467x+0.28. The formulation that was pre-blended with glyceryl behenate, in accordance with the present invention, has improved results such as, low rate of DKP formation.

TABLE 5
Co-milled ˜60 mesh

| | | | | | % LC | | | |
|---|---|---|---|---|---|---|---|---|
| Sample # | Strength | Initial | 2 wk 40/75 | 4 wk 40/75 | 8 wk 40/75 | 8 wk RT | 12 wk 40°/75 | 12 wk RT |
| 73F74A | 1.25 mg | 104.0 | 102.0 | 103.4 | 101.6 | 102.8 | 101.2 | 103.0 |
| 74F75A | 1.25 mg | 104.4 | 101.7 | 103.23 | 99.7 | 102.8 | 101.1 | 104.3 |
| 75F76A | 1.25 mg | 103.9 | 100.8 | 102.4 | 102.2 | NT | 102.2 | NT |
| | | % DKP | | | | | | |
| | | Initial | 2 wk 40/75 | 4 wk 40/75 | 8 wk 40/75 | 8 wk RT | 12 wk 40°/75 | 12 wk RT |
| 73F74A | 1.25 mg | 0.31 | 0.64 | 1.08 | 1.87 | 0.35 | 2.79 | 0.41 |
| 74F75A | 1.25 mg | 0.33 | 0.67 | 1.19 | 2.22 | 0.37 | 3.1 | 0.42 |
| 75F76A | 1.25 mg | 0.31 | 0.63 | 0.98 | 1.65 | NT | 2.48 | NT |

FIG. 3 represents a linear regression of the rate of DKP formulation of samples 73F74A and 74F75A. 73F74A is represented by the solid line and the rate of DKP % is represented by the equation y=0.0314x+0.3043. 74F75A is represented by the dashed line and the rate of DKP % is represented by the equation y=0.0286x+0.3257.

### Example 3

Tablets made from a 6 kg batch, were made according to the formulation shown in Table 6. The ramipril was pre-blended with glyceryl behenate.

TABLE 6

| | | | | sodium | sodium | |
|---|---|---|---|---|---|---|
| Sample | | Glyceryl | Microcrystalline | stearyl | carboxymethyl- | |
| 100 mg | GECoated | Behenate | cellulose | fumarate | cellulose | Co- |
| Tablets | Ramipril | (Compritol) | (Prosolve) | (PRUV ™) | (Ac-di-Sol) | milled |
| 76F74A | 1.49% w/w | 4% | 92.41% | 0.1% | 2% | 60 mesh |

The GECoated ramipril was co-milled to 60 mesh. The milled GECoated ramipril was pre-blended with glyceryl behenate. Half of half of the microcrystalline cellulose was added to a 16-quart blender along with the pre-blended ramipril and glyceryl behenate, sodium stearyl fumerate, sodium carboxymethylcellulose and the remainder of the microcrystalline cellulose. The mixture was mixed for between 15-25 minutes, then blended for between 6-10 minutes. The LC % and DKP % of sample number 76F74A is shown in Table 7.

TABLE 7

| | | % LC | | | | | |
|---|---|---|---|---|---|---|---|
| Sample # | Strength | Initial | 2 wk 40/75 | 4 wk 40/75 | 8 wk 40/75 | 12 wk 40/75 | 24 wk 40/75 |
| 76F74A | 1.25 mg | 104.4 | 102.6 | 102.7 | 100.4 | 98.33 | 98.6 |
| | | % DKP | | | | | |
| | | Initial | 2 wk 40/75 | 4 wk 40/75 | 8 wk 40/75 | 12 wk 40/75 | 24 wk 40/75 |
| 76F74A | 1.25 mg | 0.31 | 0.7 | 1.1 | 1.92 | 2.6 | 4.7 |
| | | Initial | 2 wk RT | 4 wk RT | 8 wk RT | 12 wk RT | 24 wk RT |
| | | 0.31 | | 0.38 | 0.46 | 0.52 | 0.7 |

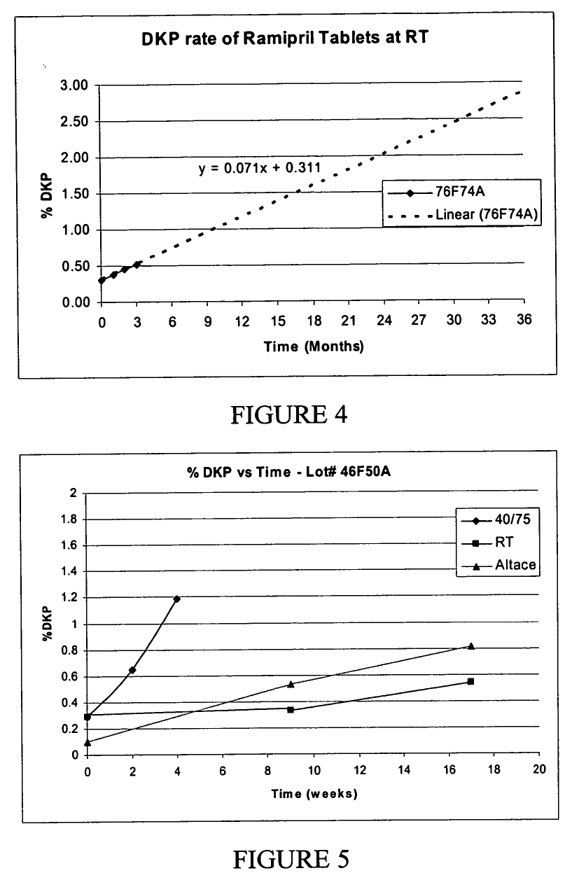

The rate of ramipril-DKP formation of sample 76F74A, made in a 6 kg batch size is graphically shown in FIG. 4.

1.25 mg, 5 mg, 10 mg and 10 mg ramipril tablets were also made according to the process used to make Batch 76F74A. The percent of ramipril-DKP formation was measured at 1 month, 3 month and 6 month at various conditions. Table 8 shows the results:

TABLE 8
Ramipril Tablet Stability DKP Assay Values

| | Ramipril, 1.25 mg | Ramipril, 5 mg | Ramipril, 10 mg | Ramipril, 20 mg |
|---|---|---|---|---|
| | Batch 040018 | Batch 040019 | Batch 040020 | Batch 040021 |
| | (DKP) RRT- | (DKP) RRT- | (DKP) RRT- | (DKP) RRT- |
| Time Point | 1.220 | 1.220 | 1.220 | 1.220 |
| Initial | 0.2476 | 0.2343 | 0.2372 | 0.2130 |
| 1 Month 25° C./60% RH | 0.3464 | 0.3352 | 0.3210 | 0.3185 |
| 1 Month 40° C./75% RH | 1.6155 | 1.3837 | 1.3987 | 1.1127 |
| 3 Month 25° C./60% RH | 0.6027 | 0.5485 | 0.5407 | 0.5138 |
| 3 Month 40° C./75% RH | 3.6725 | 3.3413 | 3.0688 | 2.5993 |
| 6 Month 25° C./60% RH | 0.8097 | 0.7152 | 0.7518 | 0.7001 |
| 6 Month 40° C./75% RH | 6.4704 | 5.8007 | 5.5618 | 4.3761 |
| 6 Month 30° C./60% RH | — | 1.7836 | 1.8951 | 1.6368 |

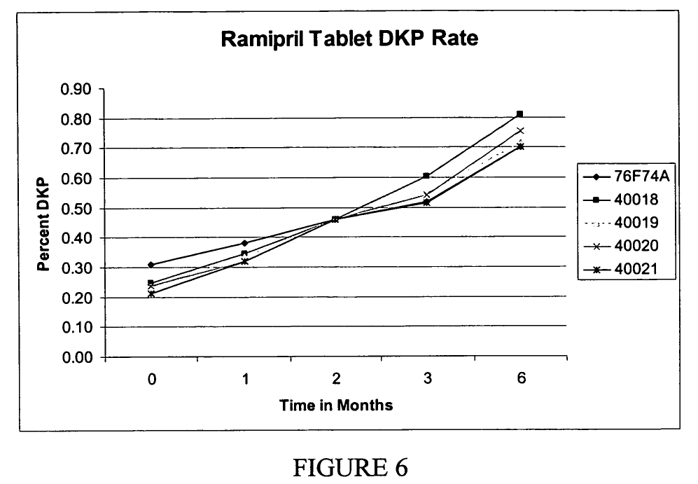

Also the rate of ramipril-DKP formation is shown in FIG. 6.

### Example 4

Direct compression tablets were prepared with the formulation as shown in Table 9. Stability data is shown in Table 10.

TABLE 9
Batch 46F50A
1.25 mg/90 mg

| Ingredients | % w/w | Mg/unit |
|---|---|---|
| GEcoated Ramipril (<150 μm) Hand-screened* | 1.66 | 1.49 |
| Silicified Microcrystalline Cellulose (Prosolv SMCC | 94.34 | 84.91 |
| 50) | | |
| Croscarmellose Sodium (Ac-Di-Sol) | 2.0 | 1.8 |
| Glyceryl Behenate (Compritol 888 ATO) | 2.0 | 1.8 |
| Total | 100 | 90 |

GECoated ramipril was pre-milled through a 60 mesh screen and then preblended with glyceryl behenate. Silicified Microcrystalline Cellulose, croscarmellose sodium and sodium stearyl fumerate were added add mixed for 20 minutes. The mixture was co-milled through a 20 mesh screen and blended for 8 minutes. The mixture was compressed into tablets.

TABLE 10

| | | | | | | | % ramipril - | | | | |
|---|---|---|---|---|---|---|---|---|---|---|---|
| | | | | % LC | | | DKP | | | | |
| | | CU | 2 wk | 4 wk | 8 wk | 12 wk | | 2 wk | 4 wk | 8 wk | 12 wk |
| Sample # | Strength | Initial | 40/75 | 40/75 | 40/75 | 40/75 | Initial | 40/75 | 40/75 | 40/75 | 40/75 |
| 46F50A | 1.25 mg | 103.6 | 102 | 101.9 | | | 0.29 | 0.65 | 1.19 | | |

As a reference dosage form Altace® was also evaluated. The results of the stability studies are graphically represented in FIG. 5. As can be seen in the graph lower levels of diketopiperazine are observed.

### Example 5

Direct compression tablets were prepared with the formulation as shown in Table 11.

TABLE 11

| | | | | | sodium | | | |
|---|---|---|---|---|---|---|---|---|
| | | | | | carboxy- | | | |
| | GE- | | Glyceryl | Microcrystalline | methyl- | | | |
| | Coated | | Behenate | cellulose | cellulose | | | |
| Sample # | Ramipril | Ramipril | (Compritol) | (Prosolve) | (Ac-di-Sol) | Ceolus | HPMC | Lactose |
| 69F70A | 1.49 | | 2 | | 2 | 45.71 | 3 | 45.80 |
| 70F71A | | 1.25 | 2 | | 2 | 45.85 | 3 | 45.90 |
| 71F72A | 1.49 | | 2 | 91.51 | 2 | | 3 | |
| 72F73A | | 1.25 | 2 | 91.75 | 2 | | 3 | |
| 62F64A | 1.49 | | 2 | | 2 | 91.51 | 3 | |
| 64F66A | | 1.25 | 2 | | 2 | 91.75 | 3 | |
| 66F68A | 1.49 | | 2 | | 2 | | 3 | 91.51 |
| 68F69A | | 1.25 | 2 | | 2 | | 3 | 91.75 |
| 69F70A | 1.49 | | 2 | | 2 | 45.71 | 3 | 45.80 |

Tablets in Table 11 were made by the following procedure. Hydroxypropylmethylcellulose (HPMC) is charged to water and mixed until fully dissolved or hydrated. Ramipril is then charged to the fluid bed processor with micro-crystalline cellulose, ceolus and lactose and spray granulated using top spray fluid bed processing. When spraying of the HPMC solution is completed the material is dried to an appropriate moisture level. The dried granules are screened and blended with the glyceryl behenate and then with sodium carboxy-methyl-celluloses. The final blend is compressed to a tablet.

Tables 12 and 13 provide levels of LC % and DKP % observed for the above formulations

TABLE 12

| | | % LC | | | | | % DKP | | | | |
|---|---|---|---|---|---|---|---|---|---|---|---|
| | | | | | | 12 wk | | | | | 12 wk |
| Lot# | Strength | Initial | 2 wk 40/75 | 4 wk 40/75 | 8 wk 40/75 | 40/75 | Initial | 2 wk 40/75 | 4 wk 40/75 | 8 wk 40/75 | 40/75 |
| 70F71A | 1.25 mg | 94.4 | 90.0 | 90.5 | 87.6 | 84.6 | 0.20 | 0.63 | 2.90 | 5.91 | 8.04 |
| 71F72A | 1.25 mg | 96.4 | 97.2 | 100.2 | 99.3 | 99.4 | 0.33 | 0.62 | 0.78 | 1.10 | 1.50 |
| 72F73A | 1.25 mg | 97.5 | NT | NT | NT | NT | 0.24 | NT | NT | NT | NT |

TABLE 13

| | | % LC | | | | | % DKP | | | | |
|---|---|---|---|---|---|---|---|---|---|---|---|
| | | | 2 wk | 4 wk | 8 wk | 12 wk | | 2 wk | 4 wk | 8 wk | 12 wk |
| Lot # | Strength | Initial | 40/75 | 40/75 | 40/75 | 40/75 | Initial | 40/75 | 40/75 | 40/75 | 40/75 |
| 62F64A | 1.25 mg | 105.7 | 106.1 | 102.5 | 100.5 | 104.6 | 0.31 | 0.85 | 0.84 | 1.24 | 1.52 |
| 64F66A | 1.25 mg | 99.8 | 96.2 | 93.8 | 87.0 | 88.1 | 0.21 | 2.37 | 3.94 | 1.27 | 9.0 |
| 66F68A | 1.25 mg | 99.4 | 98.0 | 98.2 | 97.4 | 97.9 | 0.28 | 0.55 | 0.69 | 1.17 | 1.36 |
| 68F69A | 1.25 mg | 93.7 | 91.1 | 90.9 | 89.2 | 87.4 | 0.12 | 1.42 | 2.62 | 4.31 | 6.12 |
| 69F70A | 1.25 mg | 105.0 | 104.1 | 108.4 | 105.7 | 105.6 | 0.35 | 0.61 | 0.85 | 1.24 | 1.63 |

### Example 6

Batch samples 040018 through 040021 were also used in a clinical study. The study followed a single-dose, open-label, four-period, four-treatment, crossover design and utilized a two-stage randomization process for 4 dose levels and 2 formulations. Each treatment was separated by a 2 week washout period. All treatments were administered following an overnight fast.

Thirty subjects were planned to be enrolled in the study to complete 24. Thirty subjects were enrolled, and 26 subjects completed the study. All 30 subjects were included in safety analyses, and 27 subjects were included in the pharmacokinetic analyses.

The test product was ramipril tablets manufactured by King Pharmaceuticals, Inc. Subjects randomized to Treatment A received a single oral dose of two ramipril 1.25 mg tablets (batch number 040018) taken with 240 mL of water. Subjects randomized to Treatment C received a single oral dose of one ramipril 5 mg tablet (batch number 040019) taken with 240 mL of water. Subjects randomized to Treatment E received a single oral dose of one ramipril 10 mg tablet (batch number 040020) taken with 240 mL of water. Subjects randomized to Treatment G received a single oral dose of one ramipril 20 mg tablet (batch number 040021) taken with 240 mL of water. The reference product was ALTACE® capsules manufactured by Aventis Pharmaceuticals, Inc. Subjects randomized to Treatment B received a single oral dose of two ALTACE® ramipril 1.25 mg capsules (lot number 1073176) taken with 240 mL of water. Subjects randomized to Treatment D received a single oral dose of one ALTACE® ramipril 5 mg capsule (lot number 1049756) taken with 240 mL of water. Subjects randomized to Treatment F received a single oral dose of one ALTACE® ramipril 10 mg capsule (lot number 11985) taken with 240 mL of water. Subjects randomized to Treatment H received a single oral dose of two ALTACE® ramipril 10 mg capsules (lot number 11985) taken with 240 mL of water.

Pharmacokinetic (PK) analysis was done using plasma concentrations of ramipril and ramiprilat. PK parameters included the maximum observed plasma concentration (Cₘₐₓ), time-to-maximum observed plasma concentration (Tₘₐₓ), and estimates of the area under the plasma concentration-time curve (AUC₀₋ₜ) where “t” was equal to 12 and 24 hours postdose for ramipril, and 24 and 48 hours postdose for ramiprilat. The PK parameters for ramipril and for ramiprilat were calculated from the plasma concentrations from the 27 subjects retained for PK analyses. Descriptive statistics (including arithmetic mean, standard deviation [SD], coefficient of variation [CV %], standard error of mean [SEM], number [N], geometric mean [Geom. M], median, minimum, and maximum) for plasma ramipril and ramiprilat concentrations at each time point, and for PK parameters, were tabulated by treatment.

Adverse events (AEs), vital signs, electrocardiograms (ECGs), physical examinations, and laboratory assessments (serum chemistry, hematology, and urinalysis) were evaluated in this study.

To evaluate the relative bioavailability, analyses of variance (ANOVA) were performed on the In-transformed AUC₀₋ₜ, AUC₀₋₁₂, AUC₀₋₂₄, and Cₘₐₓ for ramipril and the In-transformed AUC₀₋ₜ, AUC₀₋₂₄, AUC₀₋₄₈, and Cₘₐₓ, for ramiprilat. The ANOVA model included subject, period and formulation as fixed effects, and subject as a random effect. Ninety percent (90%) confidence intervals (CI) for the ratios of least-squares means (LSM) were derived by exponentiation of the CI obtained for the difference between treatment LSM resulting from the analyses on the In-transformed AUC₀₋ₜ, AUC₀₋₁₂, AUC₀₋₂₄, and Cₘₐₓ for ramipril and the In-transformed AUC₀₋ₜ, AUC₀₋₂₄, AUC₀₋₄₈, and Cₘₐₓ for ramiprilat. The ratios of LSM and CI were expressed as a percentage relative to the commercial capsules (ALTACE®). The comparisons of interest were Treatment A versus B, Treatment C versus D, Treatment E versus F, and Treatment G versus H. Equivalent bioavailability was to be concluded if the 90% CI for the In-transformed AUC₀₋ₜ, AUC₀₋₁₂, AUC₀₋₂₄, and Cₘₐₓ for ramipril and In-transformed AUC₀₋ₜ, AUC₀₋₂₄, AUC₀₋₄₈, and Cₘₐₓ for ramiprilat fell within the 80-125% range, where the rate and extent of exposure can be considered equivalent between the test and reference treatments.

Separately for the test (A, C, E, and G) and reference (B, D, F, and H) formulations, dose proportionality was evaluated. ANOVA were performed on the In-transformed PK parameters AUC₀₋ₜ, AUC₀₋₁₂, AUC₀₋₂₄, and Cₘₐₓ for ramipril and the PK parameters AUC₀₋ₜ, AUC₀₋₂₄, AUC₀₋₄₈, and Cₘₐₓ for ramiprilat. The ANOVA model included period as a fixed effect, intercept as a random effect, and in-transformed dose as a covariate. Ninety-five percent (95%) CI for the slope was calculated for each of the In-transformed PK parameters for plasma ramipril and ramiprilat. Dose proportionality was established if the 95% CI included the value of 1.

The ratios of LSM (with the 90% CI) derived from the analyses of the In-transformed PK parameters AUC₀₋ₜ, AUC₀₋₁₂, AUC₀₋₂₄, and Cₘₐₓ for ramipril and AUC₀₋ₜ, AUC₀₋₂₄, AUC₀₋₄₈, and Cₘₐₓ for ramiprilat, for the tablet/commercial capsule comparisons of interest are presented. In addition, the 95% CI for the slope dose proportionality results calculated for the in-transformed PK parameters AUC₀₋ₜ, AUC₀₋₁₂, AUC₀₋₂₄, and Cₘₐₓ, for ramipril and AUC₀₋ₜ, AUC₀₋₂₄, AUC₀₋₄₈, and Cₘₐₓ, for ramiprilat are presented in Tables 14 and 15.

TABLE 14
Relative Bioavailability Results for Ramipiril in Plasma for Equivalent Doses of 2.5, 5, 10, and 20 mg
Ramipril Tablets Compared to Commercial Ramipril Capsules (ALTACE ®)
Ratios of LSM (90% CI)

| | 2 × 1.25 mg tablets (A) | 5 mg tablet (C) | 10 mg tablet (E) | 20 mg tablet (G) |
|---|---|---|---|---|
| | vs. | vs. | vs. | vs. |
| Parameter | 2 × 1.25 mg capsules (B) | 5 mg capsule (D) | 10 mg capsule (F) | 2 × 10 mg capsules (H) |
| AUC₀₋ₜ | 126.9% (114.0-141.4%) | 106.9% (95.8-119.2%) | 112.0% (99.7-125.9%) | 95.8% (85.5-107.4%) |
| (ng · h/mL) | | | | |
| AUC₀₋₁₂ | 128.6% (115.1-143.6%) | 106.8% (95.8-119.1%) | 112.3% (100.0-126.2%) | 96.5% (85.4-109.1%) |
| (ng · h/mL) | | | | |
| AUC₀₋₂₄ | 129.8% (115.9-145.3%) | 107.2% (95.9-119.8%) | 112.6% (100.0-126.8%) | 96.6% (85.3-109.5%) |
| (ng · h/mL) | | | | |
| Cₘₐₓ | 117.5% (97.1-142.3%) | 107.8% (88.8-130.8%) | 105.0% (85.4-129.1%) | 93.1% (76.1-114.0%) |
| (ng/mL) | | | | |

TABLE 15
Relative Bioavailability Results for Ramiprilat in Plasma for Equivalent Doses of 2.5, 5, 10, and 20 mg
Ramipril Tablets Compared to Commercial Ramipril Capsules (ALTACE ®)
Ratios of LSM (90% CI)

| | 2 × 1.25 mg tablets (A) | 5 mg tablet (C) | 10 mg tablet (E) | 20 mg tablet (G) |
|---|---|---|---|---|
| | vs. | vs. | vs. | vs. |
| Parameter | 2 × 1.25 mg capsules (B) | 5 mg capsule (D) | 10 mg capsule (F) | 2 × 10 mg capsules (H) |
| AUC₀₋ₜ | 109.1% (101.4-117.3%) | 102.2% (94.9-110.0%) | 97.8% (90.4-105.8%) | 95.6% (88.5-103.3%) |
| (ng · h/mL) | | | | |
| AUC₀₋₂₄ | 110.6% (101.4-120.6%) | 101.4% (92.9-110.7%) | 97.1% (88.5-106.7%) | 95.0% (86.7-104.1%) |
| (ng · h/mL) | | | | |
| AUC₀₋₄₈ | 109.1% (101.4-117.3%) | 102.2% (94.9-110.0%) | 97.8% (90.4-105.8%) | 95.6% (85.5-103.3%) |
| (ng · h/mL) | | | | |
| Cₘₐₓ | 114.8% (100.4-131.2%) | 92.6% (80.8-106.0%) | 100.1% (86.6-115.7%) | 94.7% (82.2-109.1%) |
| (ng/mL) | | | | |

Of the 30 subjects dosed in this study, 15 subjects (50%) experienced a total of 38 treatment-emergent AEs: 4 subjects each following Treatments C and G; 3 subjects each following Treatments A, D, and H; 2 subjects each following Treatments B and F; and 1 subject following Treatment E. Headache and dizziness were the most common AEs reported in this study. Thirty-one (31) of the 38 AEs were mild in severity and 7 were moderate. The Investigator considered 29 of the 38 AEs to be possibly or reasonably attributable to the study treatment. No serious adverse events occurred in this study and no subject discontinued the study due to an AE. All AEs resolved by the end of the study. Notable AEs considered possibly or reasonably attributable to study drug included single AEs of vomiting, increased AST, increased ALT, and hypotension. No clinically relevant trends were observed in the clinical laboratory, vital sign, physical examination, or ECG parameters.

In the assessment of relative bioavailability, the AUCs of ramipril were comparable at dose levels of 5 and 20 mg for the tablet and commercial capsule, however, they were not comparable at the 2.5 and 10 mg dose levels. In addition, the rate of exposure (Cₘₐₓ) of ramipril was not comparable between the tablet and commercial capsule at all dose levels studied.

For the active metabolite ramiprilat, Cₘₐₓ and AUCs were comparable between the tablet and commercial capsule over the dose range studied, with the exception of Cₘₐₓ at the 2.5 mg dose, which may be due to a higher inter-subject variability (% CV approximately 25-79%) of ramiprilat plasma concentrations at that dose.

Dose proportionality within the 2.5 to 20 mg dose range could not be statistically rejected for ramiprilat AUC₀₋₂₄, since the 95% CI included the value of 1. For the tablet and commercial capsule formulations, dose proportionality could not be statistically concluded for ramipril AUC₀₋ₜ, AUC₀₋₁₂, AUC₀₋₂₄, and Cₘₐₓ and for ramiprilat AUC₀₋ₜ, AUC₀₋₄₈, and Cₘₐₓ, since the 95% CI did not include the value of 1. However, results should be interpreted with caution since the majority of statistical values were very close to the value of 1 for the 95% CI.

When looking at the descriptive PK results (geometric means) of ramipril, there seemed to be a more than proportional increase in the PK parameters AUC₀₋ₜ, AUC₀₋₁₂, and AUC₀₋₂₄ as doses were increased from 2.5 to 20 mg for the capsule and from 10 to 20 mg for the tablet formulation. The increase in the PK parameters AUC₀₋ₜ, AUC₀₋₁₂, and AUC₀₋₂₄ was proportional for ramipril from 2.5 to 5 mg for the tablet formulation. The results for the metabolite (ramiprilat) indicated a less than proportional increase for the PK parameters AUC₀₋ₜ and AUC₀₋₄₈. For the PK parameter AUC₀₋₂₄, the increase was proportional for ramiprilat as doses were increased from 2.5 to 20 mg. In addition, for both ramipril and ramiprilat, a more than proportional increase was observed for Cₘₐₓ, as doses increased from 2.5 to 20 mg. These variable results may be due to several factors: high inter-subject variability (% CV approximately 8-261%) in the plasma concentrations and the occurrence of many measurable predose concentrations greater than 5% of Cₘₐₓ for ramiprilat in Periods 2, 3, and 4, which may be due to inappropriate wash-out periods. The non-zero predose concentrations occurred most probably because of the reported long half-life of ramiprilat, which binds very tightly to the angiotensin converting enzymes (ACE), and therefore, the wash-out period may never be really long enough because of this strong binding.

Ramipril and ALTACE® administered orally in 2.5, 5, 10, and 20 mg doses appeared to be safe and generally well tolerated by the group of healthy male and female subjects in this study.

While the present invention may be embodied in many different forms, several embodiments are discussed herein with the understanding that the present disclosure is to be considered only as an exemplification of the principles of the invention, and it is not intended to limit the invention to the embodiments described or illustrated.

What is claimed:

1. A pharmaceutical composition comprising ramipril coated by a blending agent, wherein the blending agent is selected from; glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

2. The composition of claim 1, wherein the blending agent is glyceryl behenate.

3. The composition of claim 1, wherein about 50 to 100% of the ramipril is coated by the blending agent.

4. The composition of claim 1, wherein about 75 to 100% of the ramipril is coated by the blending agent.

5. The composition of claim 1, wherein about 95 to 100% of the ramipril is coated by the blending agent.

6. The composition of claim 1, wherein the blending agent is at least 0.1% by weight.

7. The composition of claim 1, wherein the blending agent is at least 1% by weight.

8. The composition of claim 1, wherein the blending agent is at least 4% by weight.

9. The composition of claim 1, wherein the ramipril is substantially stable against decomposition into a degradant product.

10. The composition of claim 9, wherein the degradant product is ramipril-diacid or ramipril-diketopiperazine.

11. The composition of claim 10, wherein the rate of decomposition of the ramipril to ramipril-diketopiperazine is less than about 0.3% by weight during about the first three months.

12. The composition of claim 10, wherein the rate of decomposition of the ramipril to ramipril-diketopiperazine is less than about 3.0% by weight during about the first thirty-six months.

13. The composition of claim 10, wherein the rate of decomposition of the ramipril to ramipril-diketopiperazine is less than about 0.09% by weight, on average, per month.

14. The composition of claim 1, wherein the ramipril is coated ramipril.

15. The composition of claim 1, wherein the composition is a solid dosage form.

16. The composition of claim 1, wherein the composition is an oral dosage form.

17. The composition of claim 1, wherein the composition is a tablet, caplet or capsule.

18. The composition of claim 17, wherein the composition is a tablet.

19. The composition of claim 1, wherein the composition further comprises an excipient.

20. The composition of claim 1, wherein the ramipril is between the amount of about 0.1 mg to 50 mg.

21. The composition of claim 1, wherein the ramipril is between the amount of about 1.25 mg to 25 mg.

22. The composition of claim 1, wherein the ramipril is between the amount of about 10 mg to 20 mg.

23. The composition of claim 1, wherein the ramipril is between the amount of about 10 or 20 mg.

24. A pharmaceutical composition comprising ramipril, wherein the ramipril is coated by a blending agent, wherein the rate of decomposition of the ramipril to ramipril-diketopiperazine is less than about 0.4% of the total weight of ramipril during the first 3 months when the pharmaceutical composition is at room temperature.

25. The composition of claim 24, wherein the rate of decomposition is about 0.3% of the total weight of ramipril during the first 3 months when the pharmaceutical composition is at room temperature.

26. The composition of claim 23, wherein the composition is a solid dosage form.

27. The composition of claim 23, wherein the composition is an oral dosage form.

28. The composition of claim 23, wherein the composition is a tablet, caplet or capsule.

29. The composition of claim 29, wherein the composition is a tablet.

30. The composition of claim 23, wherein the ramipril is between the amount of about 1.25 mg to 25 mg.

31. The composition of claim 23, wherein the ramipril is between the amount of about 10 mg to 20 mg.

32. The composition of claim 23, wherein the ramipril is in the amount of about 10 mg or 20 mg.

33. The composition of claim 23, wherein the ramipril is coated ramipril.

34. A pharmaceutical composition comprising ramipril, wherein the ramipril is coated by a blending agent, wherein the rate of decomposition of the ramipril to ramipril-diketopiperazine is less than about 0.75% of the total weight of ramipril during the first 6 months when the pharmaceutical composition is at room temperature.

35. The composition of claim 34, wherein the rate of decomposition is about 5% of the total weight of ramipril during the first 6 months when the pharmaceutical composition is at room temperature.

36. The composition of claim 34, wherein the composition is a solid dosage form.

37. The composition of claim 34, wherein the composition is an oral dosage form.

38. The composition of claim 34, wherein the composition is a tablet, caplet or capsule.

39. The composition of claim 39, wherein the composition is a tablet.

40. The composition of claim 34, wherein the ramipril is between the amount of about 1.25 mg to 25 mg.

41. The composition of claim 34, wherein the ramipril is in the amount of about 10 mg to 20 mg.

42. The composition of claim 34, wherein the ramipril is in the amount of about 10 or 20 mg.

43. The composition of claim 34, wherein the ramipril is coated ramipril.

44. A pharmaceutical composition comprising ramipril, wherein the ramipril is coated by a blending agent, wherein the rate of decomposition of the ramipril to ramipril-diketopiperazine is less than about 3.0% of the total weight of ramipril during the first 36 months when the pharmaceutical composition is at room temperature.

45. The composition of claim 44, wherein the rate of decomposition is about 2.0% of the total weight of ramipril during the first 36 months when the pharmaceutical composition is at room temperature.

46. The composition of claim 44, wherein the rate of decomposition is about 1.5% of the total weight of ramipril during the first 36 months when the pharmaceutical composition is at room temperature.

47. The composition of claim 44, wherein the composition is a solid dosage form.

48. The composition of claim 44, wherein the composition is an oral dosage form.

49. The composition of claim 44, wherein the composition is a tablet, caplet or capsule.

50. The composition of claim 49, wherein the composition is a tablet.

51. The composition of claim 44, wherein the ramipril is between the amount of about 1.25 mg to 25 mg.

52. The composition of claim 44, wherein the ramipril is between the amount of about 10 mg to 20 mg.

53. The composition of claim 44, wherein the ramipril is between the amount of about 10 mg or 20 mg.

54. The composition of claim 44, wherein the coated ramipril is coated ramipril particles.

55. A pharmaceutical composition comprising ramipril, wherein the ramipril is coated by a blending agent, wherein the rate of decomposition of the ramipril to ramipril-diketopiperazine is less than about 0.09%, on average, of the total weight of ramipril per month when the pharmaceutical composition is at room temperature.

56. The composition of claim 55, wherein the rate of decomposition is about 0.05% or less on average, of the total weight of ramipril per month when the pharmaceutical composition is at room temperature.

57. The composition of claim 55, wherein the composition is a solid dosage form.

58. The composition of claim 55, wherein the composition is an oral dosage form.

59. The composition of claim 55, wherein the composition is a tablet, caplet or capsule.

60. The composition of claim 59, wherein the composition is a tablet.

61. The composition of claim 55, wherein the ramipril is in the amount of about 1.25 mg to 25 mg.

62. The composition of claim 55, wherein the ramipril is between the amount of about 10 mg to 20 mg.

63. The composition of claim 55, wherein the ramipril is between the amount of about 10 or 20 mg.

64. The composition of claim 55, wherein the ramipril is coated ramipril.

65. A method of making a pharmaceutical composition comprising combining ramipril with a blending agent, wherein the ramipril is coated by blending agent.

66. The composition of claim 65, wherein about 50 to 100% of the ramipril is coated by the blending agent.

67. The composition of claim 65, wherein about 75 to 100% of the ramipril is coated by the blending agent.

68. The composition of claim 65, wherein about 95 to 100% of the ramipril is coated by the blending agent.

69. A method of making a pharmaceutical composition comprising first pre-blending or co-milling ramipril with a blending agent, wherein the blending agent is selected from; glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

70. The method of claim 69, further comprising adding a diluent, lubricant, disintegrant or a combination thereof.

71. The method of claim 69, further comprising compressing the ramipril with a blending agent into tablets.

72. The method of claim 69, wherein the blending agent is glyceryl behenate.

73. The method of claim 69, wherein the blending agent is at least 0.1% by weight.

74. The method of claim 69, wherein the blending agent is at least 1% by weight.

75. The method of claim 69, wherein the blending agent is at least 4% by weight.

76. The method of claim 69, wherein the ramipril is coated ramipril.

77. The method of claim 69, wherein the composition is a solid dosage form.

78. The method of claim 69, wherein the composition is an oral dosage form.

79. The method of claim 69, wherein the composition is a tablet, caplet or capsule.

80. The method of claim 79, wherein the composition is a tablet.

81. The method of claim 69, wherein the ramipril is in the amount of about 0.1 mg to 50 mg.

82. The method of claim 69, wherein the ramipril is in the amount of about 1.25 mg to 25 mg.

83. The method of claim 69, wherein the ramipril is in the amount of about 10 mg to 20 mg.

84. The method of claim 69, wherein the ramipril is in the amount of about 10 mg or 20 mg.

85. A method of making a pharmaceutical composition comprising first pre-blending and/or co-milling ramipril and glyceryl behenate; and combining the ramipril and glyceryl behenate with microcrystalline cellulose and croscarmellose sodium.

86. A product made by the process of claim 85.

87. A method of treating a cardiovascular disorders comprising administering a composition as claimed in claims 1.

88. A method of treating the cardiovascular disorder of claim 87, wherein the cardiovascular disorder is hypertension, heart failure, congestive heart failure, myocardial infarction, atherosclerotic cardiovascular disease, asymptomatic left ventricular dysfunction, chronic renal insufficiency, and diabetic or hypertensive nephropathy.
